(19) United States
(12) Patent Application Publication
(10) Pub. No.: US 2024/0215915 A1
(43) Pub. Date: Jul. 4, 2024

(54) MANUFACTURING METHOD OF MICRONEEDLE BIOSENSOR

(71) Applicant: ALBITI INC., Seoul (KR)

(72) Inventors: JUN YOUNG AHN, Palo Alto, CA (US); Eun Hee JANG, Seoul (KR)

(21) Appl. No.: 18/603,502

(22) Filed: Mar. 13, 2024

Related U.S. Application Data
(63) Continuation of application No. PCT/KR2022/000618, filed on Jan. 13, 2022.
(63) Continuation of application No. PCT/KR2022/000620, filed on Jan. 13, 2022.
(63) Continuation of application No. PCT/KR2022/001271, filed on Jan. 25, 2022.

(30) Foreign Application Priority Data
Sep. 15, 2021 (KR) 10-2021-0123253
Sep. 23, 2021 (KR) 10-2021-0125475
Sep. 23, 2021 (KR) 10-2021-0125522

(51) Int. Cl. A61B 5/00; A61B 5/262; A61B 5/268; B29C 33/40; C23C 14/20

(52) U.S. Cl. CPC A61B 5/685; A61B 5/262; A61B 5/268; B29C 33/405; C23C 14/20; A61B 2562/125

(57) ABSTRACT

The present invention provides a manufacturing method of a microneedle biosensor including a support layer including a) a step of forming a mold by forming grooves corresponding to shapes of microneedles of a working electrode, a counter electrode, and a reference electrode in a solid resin block; b) a step of imprinting the working electrode, the counter electrode, and the reference electrode using acryl or PLA on the mold; c) a step of forming a support layer by coating an epoxy- or urethane-based photo-curable adhesion after performing the step b); d) a step of forming a metal layer by forming shadow masks corresponding to patterns of the working electrode, the counter electrode, and the reference electrode and sputtering an Au or Au+Ti/Cr adhesive layer, after performing the step c); and e) forming a passivation layer on the metal layer.

### TECHNICAL FIELD

The present invention relates to a manufacturing method of a microneedle biosensor including a support layer, a manufacturing method of a microneedle biosensor using a reverse mold, and a manufacturing method of a microneedle biosensor including a passivation layer.

### BACKGROUND ART

In order to diagnose diabetes and manage the diabetes so as not to develop into complications, systematic blood sugar measurement and treatment need to be carried out together. According to the typical diabetes management, an amount of insulin to be injected is determined according to a patient's blood sugar level and the insulin is administrated at predetermined time intervals. However, the patient's blood sugar level and the change in blood sugar level in accordance with insulin administration are different for individual patients so that it is difficult to accurately and efficiently determine an amount of insulin to be injected, an injection timing, and interval.

In order to solve this problem, a continuous glucose monitoring (CGM) system may be used. The continuous glucose monitoring system was first developed by Medtronic (Minneapolis, MN, USA), approved by U. S. FDA in June 1996 and helps treat diabetic patients who have large blood sugar fluctuations and frequent hypoglycemia. The continuous glucose monitoring system is configured by three parts: a blood sugar sensor, a wireless transmitter, and a receiver. The sensor is inserted into the subcutaneous fat to measure sugar from the interstitial fluid. The latest version of continuous glucose monitoring system shows a measured blood sugar level in real time and allows appropriate action immediately.

The continuous glucose monitoring system of the related art includes a sensor which is inserted into a body to measure a blood sugar from the blood, a needle which guides the sensor to be inserted into the body, and a separate applicator coupling structure to apply the sensor module to the body. The sensor is placed in a hollow of a syringe needle, and subcutaneously pierced by the syringe needle to be inserted into the subcutaneous fat. The sensor is placed in the hollow of the syringe needle. A size of the syringe needle which is used to detect the blood sugar is at most 21 gauges. A sensing strip needs to be disposed in the hollow of the syringe needle so that diameters of the syringe needles used as the sensor needle of the continuous glucose measuring system are 600 nm to 800 nm. However, when the diameter of the sensor needle is 600 nm to 800 nm, it causes the pain to the user, which causes the discomfort during the continuous usage.

### DISCLOSURE

### Technical Problem

The present invention has been made in an effort to solve the problem of the related art and provides a method for manufacturing a microneedle biosensor which reduces the pain of the user during wearing in a minimally invasive manner.

### Technical Solution

In order to achieve the above-described object, the present invention provides a manufacturing method of a microneedle biosensor including a support layer including a) a step of forming a mold by forming grooves corresponding to shapes of microneedles of each of a working electrode, a counter electrode, and a reference electrode in a solid resin block; b) a step of imprinting the working electrode, the counter electrode, and the reference electrode using acryl or PLA on the mold; c) a step of forming a support layer by coating an epoxy- or urethane-based photo-curable adhesion after performing the step b); d) a step of forming a metal layer by forming shadow masks corresponding to patterns of the working electrode, the counter electrode, and the reference electrode and sputtering an Au or Au+Ti/Cr adhesive layer, after performing the step c); and e) forming a passivation layer on the metal layer.

The working electrode includes a first base of a circular thin film type, a plurality of microneedles which perpendicularly protrudes on the first base, and a first wiring line which extends from one end of a circumference of the first base, the counter electrode includes a second base of a strip thin film type which forms a part of a second circumference spaced apart from the circumference of the first base with a setting distance to be concentric with the first base, a plurality of microneedles which perpendicularly protrudes on the second base, and a second wiring line which extends from one end of the second base to be horizontally disposed with the first wiring line, and the reference electrode includes a third base of a strip thin film type which is spaced apart from the other end of the second base with a setting interval and forms the second circumference with the strip shape of the second base, a plurality of microneedles which perpendicularly protrudes on the third base, and a third wiring line which extends from one end of the third base.

The second base occupies ¾ of the second circumference and the third base occupies ¼ of the second circumference.

The method further includes a step of plating a tip of the microneedle of the working electrode with Pt-black and coating the tip with Nafion after performing the step d).

The method further includes a step of coating the microneedle of the reference electrode with Ag/AgCl after performing the step e).

According to another embodiment of the present invention, a manufacturing method of a microneedle biosensor includes a step S31 of forming a mold for forming a microneedle polymer layer; a step S32 of imprinting the working electrode, the counter electrode, and the reference electrode using acryl or PLA on the mold; and a step S33 of forming a metal layer by forming shadow masks corresponding to patterns of the working electrode, the counter electrode, and the reference electrode and sputtering an Au or Au+Ti/Cr adhesive layer, after performing the step S32. The step S31 includes (a) a step of forming a primary microneedle polymer layer; (b) a step of placing the primary microneedle polymer layer in a container with a mold shape; (c) a step of forming a support layer on the primary microneedle polymer layer placed in the container; (d) a step of inputting and curing a mold material polydimethylsiloxane (PDMS) after drying the support layer in the step (c); and (e) a step of completing a reverse mold by separating the PDMS mold cured in the step (d).

The working electrode includes a first base of a circular thin film type, a plurality of microneedles which perpendicularly protrudes on the first base, and a first wiring line which extends from one end of a circumference of the first base, the counter electrode includes a second base of a strip thin film type which forms a part of a second circumference spaced apart from the circumference of the first base with a setting distance to be concentric with the first base, a plurality of microneedles which perpendicularly protrudes on the second base, and a second wiring line which extends from one end of the second base to be horizontally disposed with the first wiring line, and the reference electrode includes a third base of a strip thin film type which is spaced apart from the other end of the second base with a setting interval and forms the second circumference with the strip shape of the second base, a plurality of microneedles which perpendicularly protrudes on the third base, and a third wiring line which extends from one end of the third base.

The second base occupies ¾ of the second circumference and the third base occupies ¼ of the second circumference.

The method further includes after performing the step S33, a step of forming a passivation layer on the metal layer.

According to another embodiment of the present invention, a manufacturing method of a microneedle biosensor including a passivation layer includes: a) a step of forming a mold by forming grooves corresponding to shapes of microneedles of each of a working electrode, a counter electrode, and a reference electrode in a solid resin block; b) a step of imprinting the working electrode, the counter electrode, and the reference electrode using acryl or PLA on the mold; c) forming a metal electrode layer by forming shadow masks corresponding to patterns of the working electrode, the counter electrode, and the reference electrode and sputtering an Au or Au+Ti/Cr adhesive layer; and d) forming a passivation layer on the metal electrode layer.

The step of forming a passivation layer includes; a process of forming a hole with a size smaller than the largest diameter of the microneedle in positions of the microneedles of the working electrode 110, the counter electrode 120, and the reference electrode 130 in each of a plastic adhesive tape with an adhesive layer formed on one surface of the metal electrode layer and a polyethylene terephthalate (PET) layer; a process of inserting the microneedle into a hole such that the adhesive surface of the plastic adhesive tape with the hole is in contact with a base and inserting the microneedle into a hole of the PET layer; and a process of pressurizing the PET layer with elastomer and continuing the pressurization on the heated hot plate in that state.

The working electrode includes a first base of a circular thin film type, a plurality of microneedles which perpendicularly protrudes on the first base, and a first wiring line which extends from one end of a circumference of the first base, the counter electrode includes a second base of a strip thin film type which forms a part of a second circumference spaced apart from the circumference of the first base with a setting distance to be concentric with the first base, a plurality of microneedles which perpendicularly protrudes on the second base, and a second wiring line which extends from one end of the second base to be horizontally disposed with the first wiring line, and the reference electrode includes a third base of a strip thin film type which is spaced apart from the other end of the second base with a setting interval and forms the second circumference with the strip shape of the second base, a plurality of microneedles which perpendicularly protrudes on the third base, and a third wiring line which extends from one end of the third base.

The second base occupies ¾ of the second circumference and the third base occupies ¼ of the second circumference.

Bottom surfaces of the first base, the second base, and the third base are attached onto an adhesive sheet.

The first wiring line perpendicularly extends from one end of the circumference of the first base, the second wiring line extends from one end of the second base to be horizontally disposed with the first wiring line, and the third wiring line extends from one end of the third base to be horizontally disposed with the first wiring line.

The hot plate is 80 to 200° C. and the elastomer is pressurized for 3 to 60 seconds.

### Advantageous Effects

According to the embodiment of the present invention configured as described above, a method for manufacturing a microneedle biosensor which accurately senses and is the most suitable for a skin surface shape while reducing the pain of the user during the wearing may be provided.

According to the embodiment of the present invention configured as described above, a method for manufacturing a microneedle biosensor with a high durability may be provided by avoiding stress concentration at a root of the microneedle.

### DESCRIPTION OF DRAWINGS

FIG. 1 is a view illustrating a microneedle sensor according to an embodiment of the present invention.

FIG. 2 is a flowchart illustrating a manufacturing process of a microneedle sensor according to an embodiment of the present invention.

FIG. 3 is a flowchart illustrating a thermal imprint process, in a PLA microneedle sensor manufacturing process.

FIG. 4 is a flowchart illustrating a UV imprint process, in an acrylic microneedle sensor manufacturing process.

FIG. 5 is a view for explaining a support layer forming process of a step S12 of FIG. 2.

FIG. 6 is an SEM photograph before and after forming a support layer.

FIG. 7 is a view for explaining a step S13 of FIG. 2.

FIG. 8 is a flowchart illustrating a manufacturing process of a microneedle sensor according to another embodiment of the present invention.

FIG. 9 is a view illustrating another embodiment of a mold process S31 of the microneedle biosensor manufacturing process of FIG. 8.

FIG. 10 is a view illustrating another embodiment of an imprint process S32 of the microneedle biosensor manufacturing process of FIG. 8.

FIG. 11 illustrates a metallization process S33 of the microneedle biosensor manufacturing process of FIG. 8.

FIG. 12 illustrates an effect of forming the microneedle polymer layer using the reverse mold 300.

FIG. 13 is a view illustrating a thermal imprint process of manufacturing a PLA microneedle layer in the microneedle sensor manufacturing process of FIG. 8.

FIG. 14 is a view illustrating a UV imprint process of manufacturing an acrylic microneedle layer in a microneedle sensor manufacturing process of FIG. 8.

FIG. 15 is a view for explaining a metallization process in the manufacturing process of a microneedle sensor of FIG. 8.

FIGS. 16 to 18 are views for explaining a passivation layer manufacturing process in the manufacturing process of a microneedle sensor of FIG. 8.

FIG. 19 illustrates an SEM photograph of a microneedle biosensor.

### BEST MODE

The present invention will be described in detail below with reference to the accompanying drawings. Herein, like component is denoted by like reference numeral, and repeated description and the detailed description of a known function and configuration that may make the purpose of the present invention unnecessarily ambiguous will be omitted. The embodiments of the present invention are provided for more completely explaining the present invention to those skilled in the art. Accordingly, the shape, the size, etc., of elements in the figures may be exaggerated for explicit comprehension.

The microneedle biosensor according to the embodiment of the present invention is a minimally invasive microneedle sensor. The present invention relates to a biosensor which infiltrates the skin with the microneedles to be in contact with the body fluids to monitor biological signals. The biosensor according to the embodiment of the present invention refers to a sensor which is mounted on the skin surface to continuously measure a blood sugar level during a set period to measure a blood sugar level from interstitial fluid (ISF) of the infiltrated host but is not limited thereto.

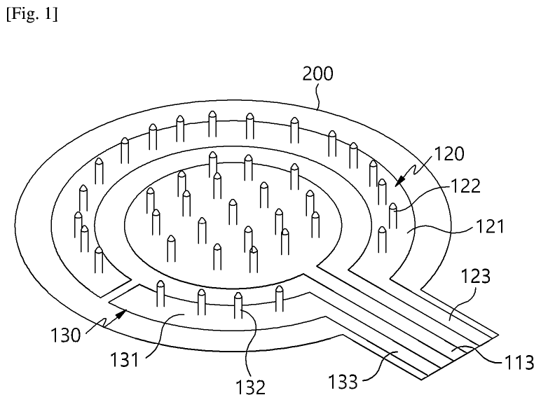

FIG. 1 is a view illustrating a microneedle sensor according to an embodiment of the present invention. As illustrated in the drawing, the microneedle sensor includes a working electrode (WE) 110, a counter electrode (CE) 120, a reference electrode (RE) 130, and an adhesive sheet 200. The working electrode 110 includes a first circular base 111, a plurality of microneedles 112 which perpendicularly protrudes on the first base 111, and a first wiring line 113 which perpendicularly extends from one end of the first base 111. The counter electrode 120 includes a second base 121 which is formed as a ¾ circular strip thin film with a setting width spaced apart from a circumference of the first base 111 with a setting interval to be concentric with the first base 111, a plurality of microneedles 122 which perpendicularly protrudes on the second base 121, and a second wiring line 123 which extends from one end of the second base 121 to be horizontally disposed with the first wiring line 113. The reference electrode 130 includes a third base 131 which is spaced apart from the other end of the second base 121 with a setting interval and is formed as a ¼ circular strip thin film with a setting width spaced apart from the circumference of the first base 111 with a setting interval to be concentric with the first base 111, a plurality of microneedles 132 which perpendicularly protrudes on the third base 131, and a third wiring line 133 which perpendicularly extends from one end of the third base 131 to be horizontally disposed with the first wiring line 113.

The counter electrode 120 and the reference electrode 130 are spaced apart from the working electrode 110 with a setting interval to enclose the working electrode 110. The working electrode 110, the counter electrode 120, and the reference electrode 130 are attached onto the adhesive sheet 200. The adhesive sheet 200 is desirably formed by applying an adhesion on one surface of a fiber or polymer sheet. The adhesive sheet 200 desirably has an elasticity in the sheet itself. The working electrode 110, the counter electrode 120, and the reference electrode 130 are attached on a surface of the adhesive sheet 200 applied with an adhesion which is attachable to the skin. The circular working electrode 110 and the counter electrode 120 and the reference electrode 130 which are spaced apart from the working electrode 110 to have a strap shape and enclose the working electrode 110 are provided and the working electrode 110, the counter electrode 120, and the reference electrode 130 are attached to the fiber or polymer sheet. Accordingly, the working electrode 110, the counter electrode 120, and the reference electrode 130 ensure a sufficient effective area for sensing. Further, when the working electrode 110, the counter electrode 220, and the reference electrode 130 are attached to the human skin which cannot be flat due to its structure, the working electrode 110, the counter electrode 120, and the reference electrode 130 are flexibly inclined according to the angle of the skin to be closely attached onto a skin contact surface. That is, when a sensor with a flat base is attached onto a skin, rather than a flat surface, edges are lifted due to resilience as time passes after attachment. However, the microneedle biosensor according to the embodiment of the present invention may solve the problem.

The microneedle biosensor according to the embodiment of the present invention is formed by sequentially laminating a polymer layer, a metal electrode layer, and a passivation layer.

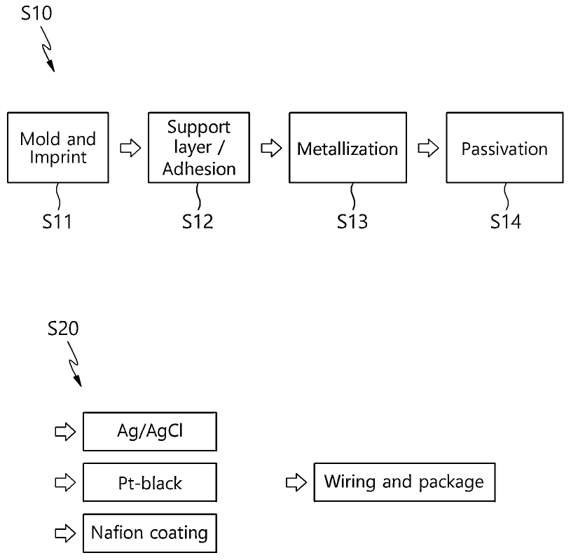

FIG. 2 is a flowchart illustrating a microneedle sensor manufacturing process illustrated in FIG. 1. As illustrated in the drawing, the method for manufacturing a microneedle biosensor includes a microneedle manufacturing process S10 configured by a mold and imprint process S11, a support layer forming process S12, a metallization process S13, and a passivation process S14 and a post-processing process S20 configured by Ag/AgCl, Pt-black, and Nafion coating and wiring and packaging processes.

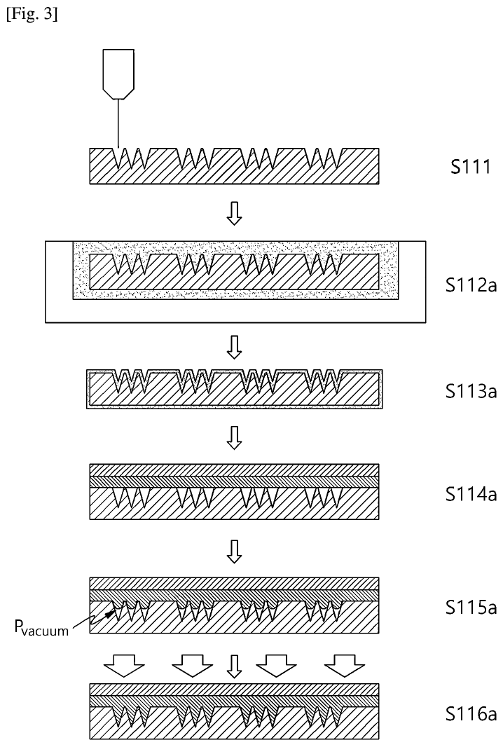

FIG. 3 is a flowchart illustrating a thermal imprint process S11 of manufacturing a PLA microneedle in the microneedle sensor manufacturing process of FIG. 2.

As illustrated in the drawing, the process is configured by a mold manufacturing step S111 of forming a groove having a shape corresponding to a microneedle on a polytetrafluoroethylene (PTFE) block with laser, a release agent coating step S112a of coating a release agent on the mold, a step S113a of drying the release agent, a step S114a of forming the PLA layer on the mold with the groove and pressurizing with ceramic, a step S115a of baking in a vacuum oven at 200° C., and a step S116a of pressurizing with a press after vacuum off. A PLA (poly lactic acid) microneedle, which is eco-friendly, non-toxic, biodegradable, and biocompatible, is formed. The PLA needle has a high elastic modulus and bucking stiffness.

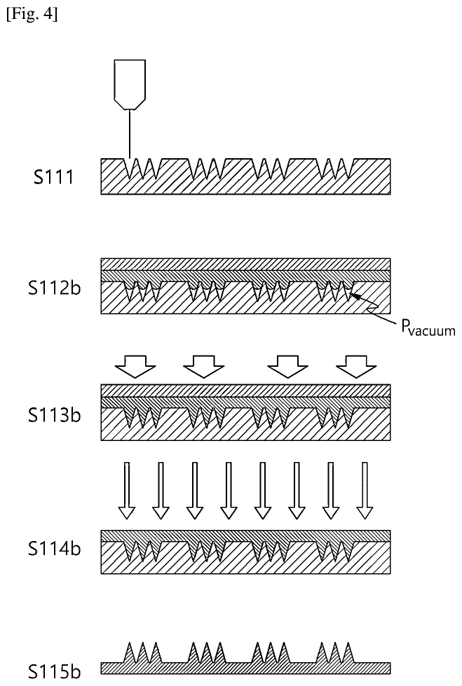

FIG. 4 is a flowchart illustrating a UV imprint process S11 of manufacturing an acrylic microneedle in a microneedle sensor manufacturing process of FIG. 2. The process includes a mold manufacturing step S111 of forming a groove having a shape corresponding to a needle on a polytetrafluoroethylene (PTFE) block with laser, a step S112b of placing an acrylic UV resin on the mold in a vacuum state, a step S113b of pressurizing with a press after vacuuming off, a UV curing step S114b, and a demolding step S115b. The acrylic microneedle has the advantage of having a short manufacturing process of approximately 5 to 10 minutes, and an acrylic microneedle has the advantage of good adhesiveness to Au.

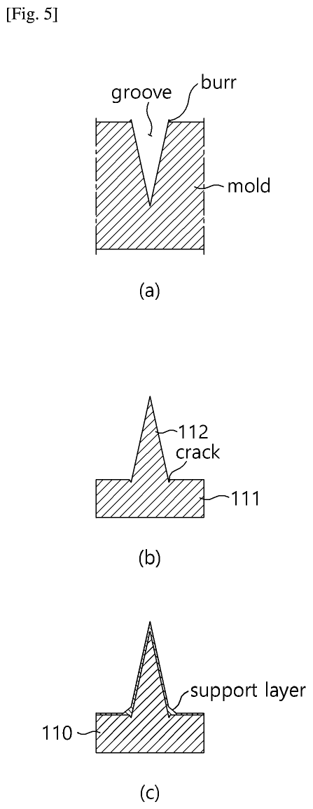

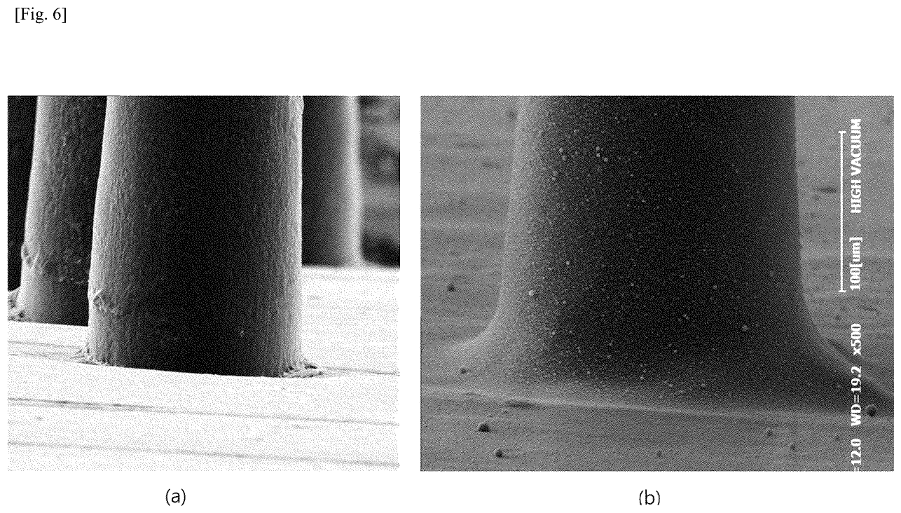

FIG. 5 is a view for explaining a support layer forming process of step S12 of FIG. 2 and FIG. 6 is an SEM photograph before and after forming a support layer.

Microneedles 112, 122, and 132 which perpendicularly protrudes on the bases 111, 121, and 131 have a problem in that a durability is weak due to a stress concentrated in root areas. Further, the needles formed of a polymer layer have a rough surface so that there may be a problem in the attachment of Au in a metal, specifically, Au deposition process. Therefore, according to the embodiment of the present invention, a support layer is coated on the polymer layer to improve an adhesiveness of the metal thin film and suppress the degradation of the durability due to the crack which may be caused in the needle root area.

FIG. 5(a) illustrates a mold, FIG. 5(b) illustrates a polymer layer separated from the mold after imprinting, and FIG. 5(c) illustrates a state in which a support layer is coated on the polymer layer. In FIG. 5, the working electrode 110 will be described as an example and the description of the counter electrode 120 and the reference electrode 130 is the same as the working electrode 110 so that the description thereof will be omitted. As illustrated in FIG. 5(a), when a groove corresponding to a shape of the microneedle is formed in a solid resin block by means of a laser processing, burrs are formed in an inlet of the groove. As illustrated in FIG. 5(b), cracks are generated around the root of the needle 112 due to the burrs. There is a problem in that the stress is concentrated in the root portion of the needle 112 due to the cracks so that the durability is weakened. Further, there is a problem in that a metal electrode layer which is laminated on the polymer layer is disconnected in the position of the crack.

According to the embodiment of the present invention, the support layer is formed on the polymer layer to solve the above-described problem. FIG. 5(c) is a schematic view illustrating a state in which the support layer is formed. The support layer is formed by coating an epoxy- or urethane-based resin or a photo-curable adhesion on the polymer layer. As the photo-curable adhesion, NOA 60, NOA 61, NOA 68 may be used. The support layer is formed by spin-coating and optically or thermally curing the epoxy- or urethane-based resin or the photo-curable adhesion on the polymer layer. The thickness is adjusted by an appropriate spin condition for every material. FIG. 6(a) illustrates a polymer layer in which a microneedle is formed before forming the support layer. As illustrated in the drawings, since a portion near the root of the microneedle perpendicularly protrudes from the base, thereafter, when the metal electrode layer is formed, the metal thin film is cut near the root to be disconnected. Further, when cracks are formed near the root, specifically, the metal thin film formed thereafter may be more easily disconnected due to the movement of the needle. FIG. 6(b) illustrates a state in which a support layer is coated on the polymer layer. As illustrated in the drawing, the support layer is smoothly connected from the base to the needle to be rounded so that problems such as the stress concentration or disconnection of the metal thin film may be suppressed. Further, the support layer may compensate for cracks in the needle root area and suppress the stress concentration and smooths the rough surface to improve the adhesiveness of the metal electrode layer.

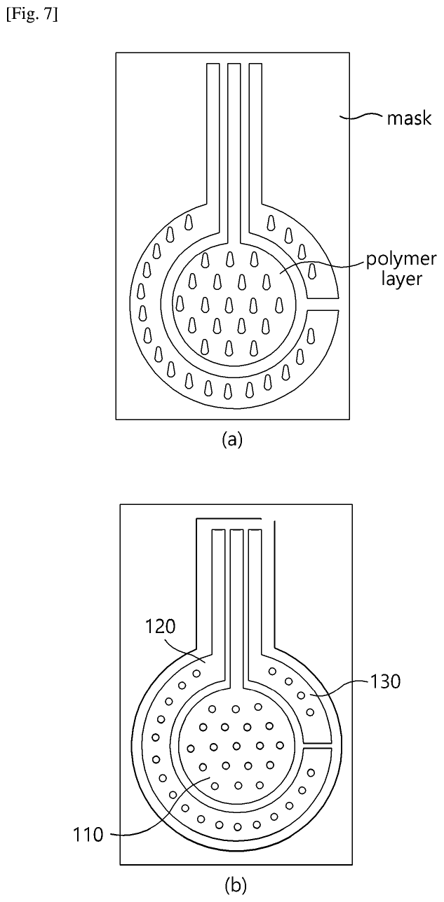

FIG. 7 is a view for explaining a metallization process S13 in the manufacturing process of a microneedle sensor of FIG. 2. In the metallization process, a shadow mask corresponding to patterns of the working electrode 110, the counter electrode 120, and the reference electrode 130 of FIG. 1 is formed on the microneedle polymer layer manufactured by the imprint process of FIG. 3 or 4 and an Au or Au+Ti/Cr adhesive layer is sputtered to form a metal layer. The metal layer serves as an electrode.

After performing the metallization process S13, a passivation process S14 is performed. The passivation process is to form a passivation layer on a metal layer. The passivation layer is an insulating layer which limits an exposed area of the metal layer to react. In the passivation process, the UV adhesion is spin-coated on the Au surface excluding a reaction area of the microneedle and then dried to form the passivation layer. As the UV adhesion, NOA 68 is desirably applied.

Thereafter, the post-processing process S20 is performed. The post-processing process S20 includes a process of coating Ag/AgCl, Pt black, and Nafion as a sensing material and a wiring and packaging process.

In a microneedle biosensor according to the embodiment of the present invention, it is desirable to coat the working electrode of the microneedle with Pt black using a glucose oxidation catalyst to measure a blood sugar. The plating is most desirably performed at a current of 5 mA using 2.5 mmol chloroplatinic acid and 0.1 M HCl.

Nafion is a biocompatible material which acts as a shield to limit the access of various in-vivo signal interference materials to Pt black. Nafion is desirably coated using a spin coating method.

The reference electrode may be formed by drop-casting the Ag/AgCl gel in the region of the reference electrode 130. In the post-processing process S20, the region of the working electrode 110 is desirably coated with Pt-black and Nafion and the reference electrode 130 is desirably coated with Ag/AgCl.

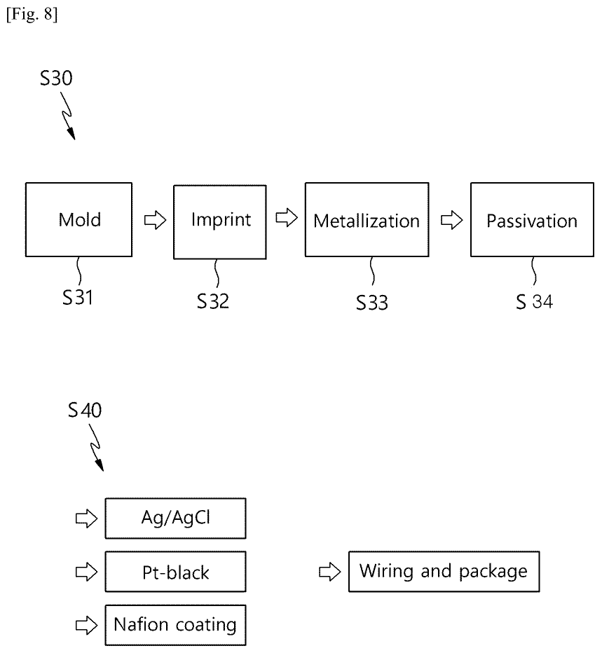

A microneedle biosensor manufacturing method using a reverse mold according to another embodiment of the present invention will be described with reference to FIGS. 8 to 13.

FIG. 8 is a flowchart illustrating a manufacturing process of a microneedle sensor according to another embodiment of the present invention.

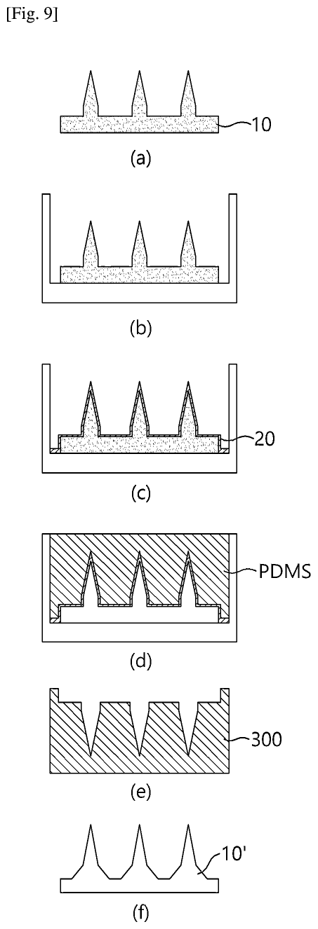

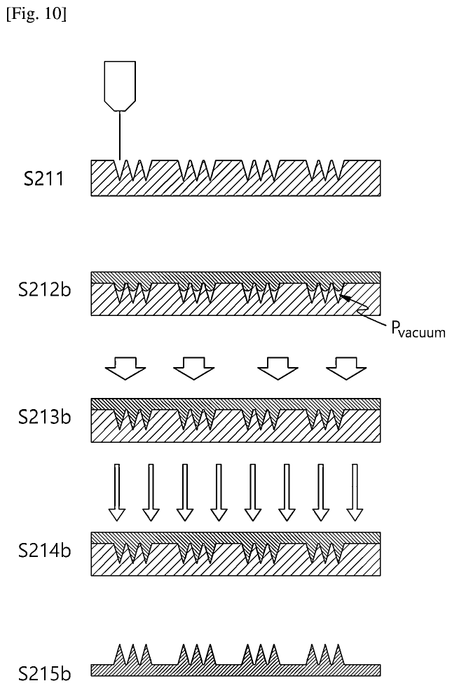

FIG. 9 is a view illustrating another embodiment of a mold process S31 of the microneedle biosensor manufacturing process of FIG. 8. The mold process S31 includes (a) a step of forming a primary microneedle polymer layer 10, (b) a step of placing the primary microneedle polymer layer 10 in a container with a mold shape, (c) a step of forming a support layer 20 on the primary microneedle polymer layer 10 placed in the container, (d) a step of inputting and curing polydimethylsiloxane (PDMS) which is a mold material after drying the support layer 20, and (e) a step of completing a reverse mold 300 by separating the PDMS mold cured in the previous step. A step (f) is a step of forming a final microneedle polymer layer 10′. In this step, after manufacturing the reverse mold 300 by the mold process of S31, the final microneedle polymer layer 10′ is formed by the imprint process of FIG. 10.

FIG. 10 illustrates an imprint process of manufacturing an acrylic microneedle and an imprint process using an acrylic UV resin in a microneedle biosensor manufacturing process. The process includes a step S212b of placing an acrylic UV resin on the reverse mold 300 manufactured in the mold manufacturing step S31 in a vacuum state, a step S213b of pressurizing with a press after vacuuming off, a UV curing step S214b, and a demolding step S215b. The acrylic microneedle has the advantage of having a short manufacturing process of approximately 5 to 10 minutes, and an acrylic microneedle has the advantage of good adhesiveness to Au.

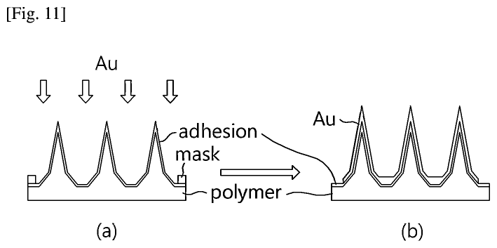

FIG. 11 is a view illustrating a metallization process in the manufacturing process of a microneedle biosensor of FIG. 8. During the metallization process, an adhesion is applied on a final microneedle polymer layer 10′ manufactured by the imprint process of FIG. 10, a shadow mask corresponding to the patterns of the working electrode 110, the counter electrode 120, and the reference electrode 130 of FIG. 1 is formed (FIG. 7(a)), and an Au or Au+Ti/Cr adhesive layer is sputtered to form a metal layer (b), to form the working electrode 110, the counter electrode 120, and the reference electrode 130.

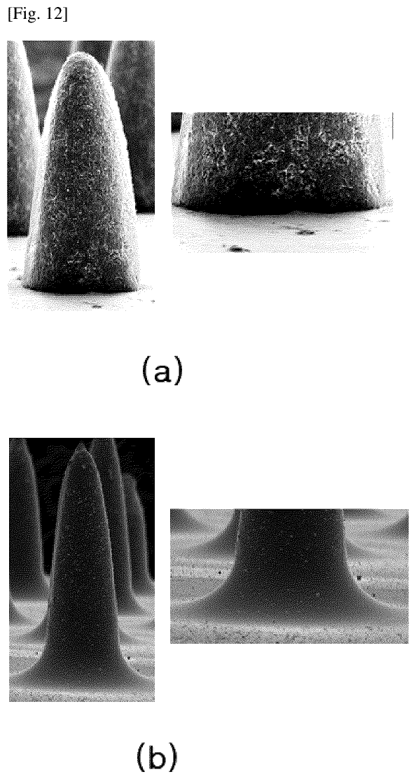

FIG. 12 illustrates an effect of forming the microneedle polymer layer using the reverse mold 300 as described above. FIG. 12(a) illustrates a microneedle polymer layer of the related art manufactured using a mold formed by laser etching and FIG. 12(b) illustrates a microneedle polymer layer according to an embodiment of the present invention. As illustrated in the drawing, in the microneedle polymer layer of the related art of FIG. 12(a), the needle perpendicularly protrudes from the base so that the stress is concentrated on the needle root to cause damages to the needle or the metal electrode layer which is deposited on the microneedle biosensor is cut to cause a problem in accurate sensing. In contrast, when the microneedle polymer layer 10′ is formed using the reverse mold 300 manufactured according to the embodiment of the present invention, the periphery of the needle root is reinforced to increase the durability of the microneedle biosensor.

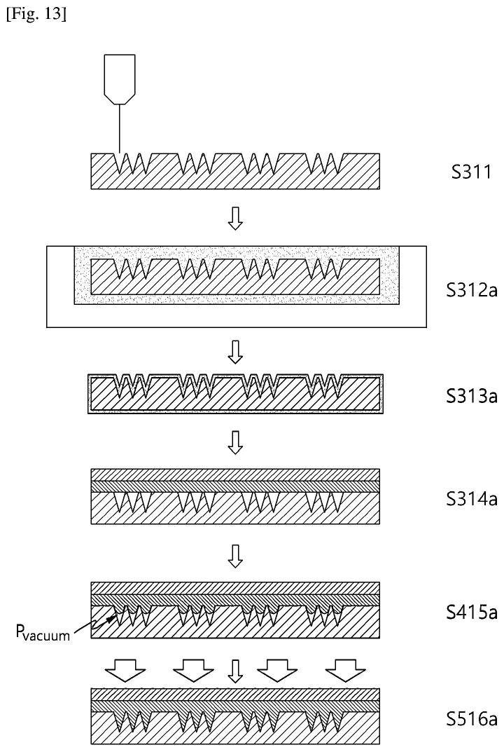

A manufacturing method of a microneedle biosensor including a passivation layer according to another embodiment of the present invention will be described with reference to FIGS. 13 to 18.

FIG. 13 is a view for explaining a thermal imprint process S31 of manufacturing a PLA microneedle layer in the microneedle sensor manufacturing process of FIG. 8. As illustrated in the drawing, the process is configured by a mold manufacturing step S311 of forming a groove having a shape corresponding to a needle on a polytetrafluoroethylene (PTFE) block with laser, a release agent coating step S312a of coating a release agent on the mold, a step S313a of drying the release agent, a step S314a of forming the PLA layer on the mold with the groove and pressurizing with ceramic, a step S315a of baking in a vacuum oven at 200° C., and a step S316a of pressurizing with a press after vacuum off. A PLA (poly lactic acid) microneedle, which is eco-friendly, non-toxic, biodegradable, and biocompatible, is formed. The PLA needle has a high elastic modulus and bucking stiffness.

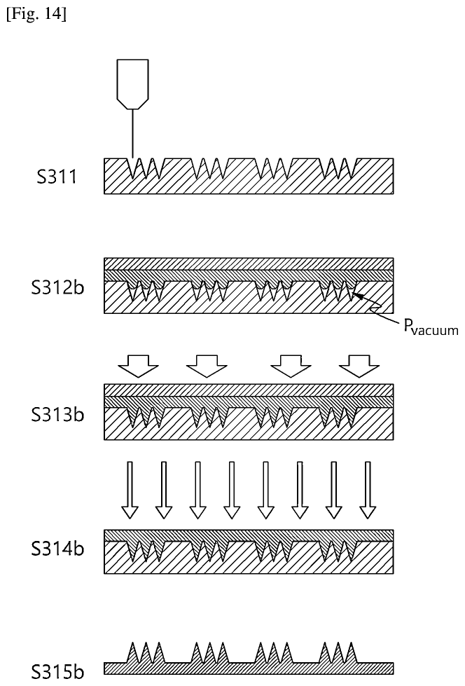

FIG. 14 is a view for explaining a UV imprint process S32 of manufacturing an acrylic microneedle layer in a microneedle sensor manufacturing process of FIG. 8. The process includes a mold manufacturing step S311 of forming a groove having a shape corresponding to a needle on a polytetrafluoroethylene (PTFE) block with laser, a step S312b of placing an acrylic UV resin on the mold in a vacuum state, a step S313b of pressurizing with a press after vacuuming off, a UV curing step S314b, and a demolding step S315b. The acrylic microneedle has the advantage of having a short manufacturing process of approximately 5 to 10 minutes, and an acrylic microneedle has the advantage of good adhesiveness to Au.

FIG. 7 is a view for explaining a metallization process S33 in the manufacturing process of a microneedle sensor of FIG. 8. In the metallization process, a shadow mask corresponding to patterns of the working electrode 110, the counter electrode 120, and the reference electrode 130 of FIG. 1 is formed on the polymer microneedle layer manufactured by the imprint process of FIG. 13 or 14 and an Au or Au+Ti/Cr adhesive layer is sputtered to form a metal electrode layer.

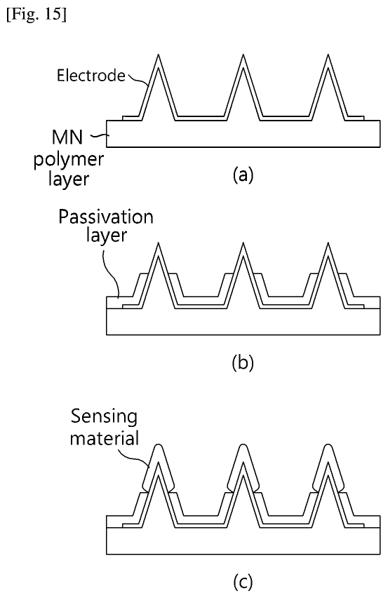

FIGS. 15 to 18 are views for explaining a passivation layer manufacturing process S34 in the manufacturing process of a microneedle sensor of FIG. 8. The passivation layer refers to an insulating layer which is formed to define an area which is exposed to sense on a base layer on the metal electrode layer. A noise which may be caused in the base area by the contact of a sensing material may be suppressed.

FIG. 15 is a view for explaining a process after forming a metal electrode layer and a sensing layer is formed on an electrode exposed after forming the passivation layer on the metal electrode layer.

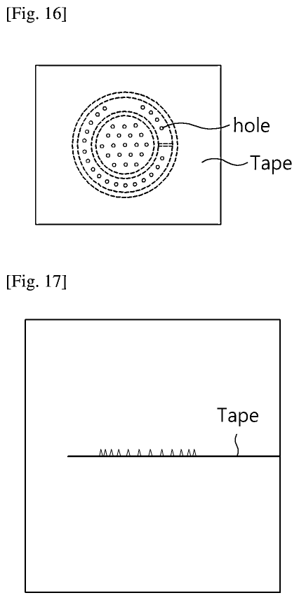

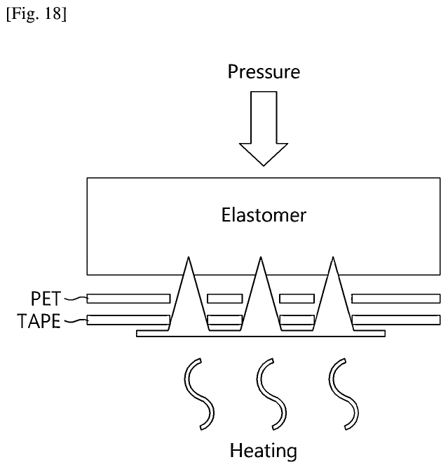

The passivation layer forming process is configured by a process of forming a hole with a smaller size than a largest diameter of the microneedle in a position of the microneedle of the working electrode 110, the counter electrode 120, and the reference electrode 130, in each of a plastic adhesive tape with an adhesive layer on one surface of the metal electrode layer and a polyethylene terephthalate (PET) layer, as illustrated in FIG. 16, a process of inserting a microneedle into a hole such that the adhesive surface of the plastic adhesive tape with a hole to be in contact with the base and inserting the microneedle into a hole of the PET layer as illustrated in FIG. 17, and a process of pressurizing the PET layer with elastomer and continuing the pressurization for 3 to 60 seconds on the heated hot plate in that state as illustrated in FIG. 18.

The hole is desirably formed using the laser patterning process.

The hot plate is desirably 80 to 200° C.

Thereafter, the post-processing process S20 is performed.

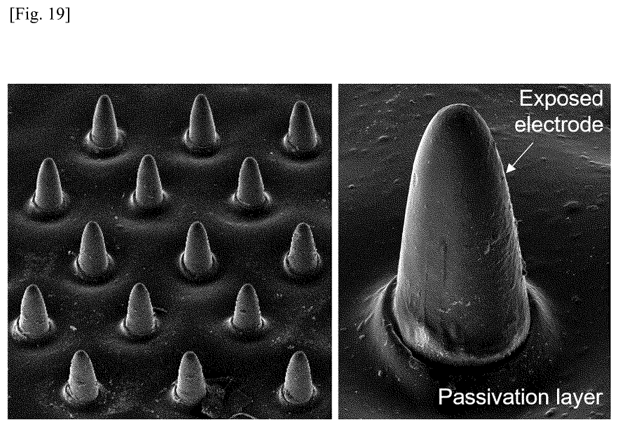

FIG. 19 illustrates an SEM photograph of a microneedle biosensor in which the process of forming the passivation layer as described above is completed. As illustrated in the drawing, the insulation is completed excluding the sensing area so that the noise may be suppressed during the sensing.

1. A manufacturing method of a microneedle biosensor including a support layer, comprising:
a) a step of forming molds by forming grooves corresponding to shapes of microneedles of a working electrode, a counter electrode, and a reference electrode in a solid resin block;
b) a step of imprinting the working electrode, the counter electrode, and the reference electrode using acryl or PLA on the mold;
c) a step of forming the support layer by coating an epoxy- or urethane-based photo-curable adhesion after performing the step b);
d) a step of forming a metal layer by forming shadow masks corresponding to patterns of the working electrode, the counter electrode, and the reference electrode and sputtering an Au or Au+Ti/Cr adhesive layer, after performing the step c); and
e) forming a passivation layer on the metal layer.

2. The manufacturing method of a microneedle biosensor including a support layer of claim 1, wherein the working electrode includes a first base of a circular thin film type, a plurality of microneedles which perpendicularly protrudes on the first base, and a first wiring line which extends from one end of a circumference of the first base,
the counter electrode includes a second base of a strip thin film type which forms a part of a second circumference spaced apart from the circumference of the first base with a setting distance to be concentric with the first base, a plurality of microneedles which perpendicularly protrudes on the second base, and a second wiring line which extends from one end of the second base to be horizontally disposed with the first wiring line, and
the reference electrode includes a third base of the strip thin film type which is spaced apart from the other end of the second base with a setting interval and forms the second circumference with a strip shape of the second base, a plurality of microneedles which perpendicularly protrudes on the third base, and a third wiring line which extends from one end of the third base.

3. The manufacturing method of a microneedle biosensor including a support layer of claim 2, wherein the second base occupies ¾ of the second circumference and the third base occupies ¼ of the second circumference.

4. The manufacturing method of a microneedle biosensor including a support layer of claim 1, further comprising:
after performing the step d), a step of coating plating a tip of the microneedle of the working electrode with Pt-black and coating the tip with Nafion.

5. The manufacturing method of a microneedle biosensor including a support layer of claim 1, further comprising:
after performing the step e), a step of coating the microneedle of the reference electrode with Ag/AgCl.

6. A manufacturing method of a microneedle biosensor using a reverse mold, comprising:
a step S11 of forming a mold for forming a microneedle polymer layer;
a step S12 of imprinting a working electrode, a counter electrode, and a reference electrode using acryl or PLA on the mold; and
a step S13 of forming a metal layer by forming shadow masks corresponding to patterns of the working electrode, the counter electrode, and the reference electrode and sputtering an Au or Au+Ti/Cr adhesive layer, after performing the step S12,
wherein the step a) includes:
(a) a step of forming a primary microneedle polymer layer;
(b) a step of placing the primary microneedle polymer layer in a container with a mold shape;
(c) a step of forming a support layer on the primary microneedle polymer layer placed in the container;
(d) a step of inputting and curing polydimethylsiloxane (PDMS) which is a mold material after drying the support layer in the step (c); and
(e) a step of completing the reverse mold by separating the PDMS mold cured in the step (d).

7. The manufacturing method of a microneedle biosensor using a reverse mold of claim 6, wherein the working electrode includes a first base of a circular thin film type, a plurality of microneedles which perpendicularly protrudes on the first base, and a first wiring line which extends from one end of a circumference of the first base,
the counter electrode includes a second base of a strip thin film type which forms a part of a second circumference spaced apart from the circumference of the first base with a setting distance to be concentric with the first base, a plurality of microneedles which perpendicularly protrudes on the second base, and a second wiring line which extends from one end of the second base to be horizontally disposed with the first wiring line, and
the reference electrode includes a third base of the strip thin film type which is spaced apart from the other end of the second base with a setting interval and forms the second circumference with a strip shape of the second base, a plurality of microneedles which perpendicularly protrudes on the third base, and a third wiring line which extends from one end of the third base.

8. The manufacturing method of a microneedle biosensor using a reverse mold of claim 7, wherein the second base occupies ¾ of the second circumference and the third base occupies ¼ of the second circumference.

9. The manufacturing method of a microneedle biosensor using a reverse mold of claim 6, further comprising:
after performing the step S13, a step of forming a passivation layer on the metal layer.

10. A manufacturing method of a microneedle biosensor including a passivation layer, comprising:
a) a step of forming a mold by forming grooves corresponding to shapes of microneedles of a working electrode, a counter electrode, and a reference electrode in a solid resin block;
b) a step of imprinting the working electrode, the counter electrode, and the reference electrode using acryl or PLA on the mold;
c) a step of forming a metal electrode layer by forming shadow masks corresponding to patterns of the working electrode, the counter electrode, and the reference electrode and sputtering an Au or Au+Ti/Cr adhesive layer; and
d) a step of forming the passivation layer on the metal electrode layer,
wherein the step of forming the passivation layer includes;
a process of forming a hole with a size smaller than a largest diameter of the microneedle in positions of the microneedles of the working electrode, the counter electrode, and the reference electrode in each of a plastic adhesive tape with an adhesive layer formed on one surface of the metal electrode layer and a polyethylene terephthalate (PET) layer;
a process of inserting the microneedle into the hole such that the adhesive surface of the plastic adhesive tape with the hole is in contact with a base and inserting the microneedle into the hole of the PET layer; and
a process of pressurizing the PET layer with elastomer and continuing the pressurization on a heated hot plate in that state.

11. The manufacturing method of a microneedle biosensor including a passivation layer of claim 10, wherein the working electrode includes a first base of a circular thin film type, a plurality of microneedles which perpendicularly protrudes on the first base, and a first wiring line which extends from one end of a circumference of the first base,
the counter electrode includes a second base of a strip thin film type which forms a part of a second circumference spaced apart from the circumference of the first base with a setting distance to be concentric with the first base, a plurality of microneedles which perpendicularly protrudes on the second base, and a second wiring line which extends from one end of the second base to be horizontally disposed with the first wiring line, and
the reference electrode includes a third base of the strip thin film type which is spaced apart from the other end of the second base with a setting interval and forms the second circumference with a strip shape of the second base, a plurality of microneedles which perpendicularly protrudes on the third base, and a third wiring line which extends from one end of the third base.

12. The manufacturing method of a microneedle biosensor including a passivation layer of claim 11, wherein the second base occupies ¾ of the second circumference and the third base occupies ¼ of the second circumference.

13. The manufacturing method of a microneedle biosensor including a passivation layer of claim 10, wherein the hot plate is 80 to 200° C. and the elastomer is pressurized for 3 to 60 seconds.
